(19) United States
(12) Patent Application Publication
(10) Pub. No.: US 2023/0313248 A1
(43) Pub. Date: Oct. 5, 2023

(54) SYNTHESIS OF C-GLYCOSIDES OF INTEREST

(71) Applicant: CarboCode S.A., Cantanhede (PT)

(72) Inventors: Ferenc Horvath, Pilisszentkereszt (HU); Györgyi Osztrovszky, Kisvarda (HU); Gyula Dekany, Queensland (AU); Agathe Bronikowski, Adelsheim (DE); Piroska Kovacs-Penzes, Jaszbereny (HU); Rafael Soares, Tamengos (PT); Jorge Santos, Alenquer (PT); Fabio Pereira, Cacém (PT); Osama Mahmoud, Konstanz (DE); Nagy Csaba, Konstanz (DE); Dário Jorge Silva Neves, Louriçal (PT)

(21) Appl. No.: 17/801,644

(22) PCT Filed: Feb. 24, 2022
(86) PCT No.: PCT/EP2021/054511

§ 371 Date: Aug. 23, 2022

(30) Foreign Application Priority Data
Feb. 24, 2020 (CH) 00218/20

(51) Int. Cl. C12P 17/16; C07H 7/02; C12N 15/70; C12N 9/10

(52) U.S. Cl. CPC C12P 17/162; C07H 7/02; C12N 9/1081; C12N 15/70; C12Y 204/99006; C12Y 204/99008; C12N 2800/101

(57) ABSTRACT

The present invention relates to a biotechnological method for producing a C-glycoside of interest. The invention further relates to the use of a sialylated C-glycoside as a donor in an enzymatic glycosylation reaction. The present invention further relates to the following C- glycosides.

### FIELD OF THE INVENTION

The present invention relates to a method for producing C-glycosides of interest using a biotechnological approach.

### BACKGROUND OF THE INVENTION

C-Glycosides are a class of compounds where a carbohydrate is bound to an aglycone or another carbohydrate via a C—C bond linkage instead of the usual C—O glycosidic linkage.

The conversion of the anomeric acetal into an ether linkage renders C-glycosides remarkably stable towards hydrolysis.

Chemical synthesis is required for the installation of the glycosidic C—C bond onto a carbohydrate unit. Several methods are available for the preparation of C-glycoside derivatives of mono- or di-saccharides (Y. Yang, B. Yu, Chem. Rev. 2017, 117, 12281-12356). These methods provide easy access to simple C-glycosides which can be used as building blocks for the preparation of complex C-oligosaccharides.

C-Oligosaccharides in which one or more glycosidic linkages have been replaced with a non-hydrolysable C—C bond represent an important class of glycomimetics and hold great promise as therapeutics and as tools for the study of key biological processes. For instance, they can be used as model compounds in enzymatic and metabolic studies (S. Howard, S. G. Withers, J. Am. Chem. Soc 1998, 120, 10326-10331), they have potential as antidiabetic (K. K. G. Ramakrishna, V. D. Tripathi, R. P. Tripathi, Trends Carbohydr. Res. 2018, 10, 10-27) and immunoactive agents (A. S. Altiti, X. Ma, L. Zhang, Y. Ban, R. W. Franck, D. R. Mootoo, Carbohydr. Res. 2017, 443-444, 73-77) they can act as enzyme inhibitors (R. R. Schmidt, D. Hansjoerg, Angew. Chem.Int. Ed. Engl. 1991, 30, 1328-1329) and have found cosmetic applications (US 2004/0048785 A1).

Despite their potential, the use of C-oligosaccharides in fundamental and clinical research is impaired by their availability. In fact, the preparation of C-oligosaccharides in significant amounts represents a challenge.

Processes for the preparation of C-oligosaccharides that are based on chemical and enzymatic synthesis exist. However, these approaches possess several limitations.

Common challenges connected to chemical synthesis are the control of stereo- and regiochemistry, the need of multiple protecting group manipulations, difficult purification and scale-up.

The enzymatic synthesis of C-oligosaccharides has been described (S. Howard, S. G. Withers, J. Am. Chem. Soc 1998, 120, 10326-10331; J. K. Fairweather, R. V. Stick, S. G. Withers, Aust. J. Chem. 2000, 53, 913-916). With this method a C-glycoside building block is elongated via sequential glycosylation catalysed by glycosynthases. Limitations to this approach include engineering the expression of and isolating the pure enzyme, as well as low yields.

### SUMMARY OF THE INVENTION

The inventors have established for the first time a biotechnological route for producing complex C-glycosides.

The present invention relates to a method for producing a C-glycoside of interest, the method comprising the steps of:
a) Providing an exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes, and wherein the exogenous precursor is a compound of General Formula I

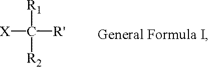

wherein
   X represents a glycosyl moiety;
   C is a carbon atom connected via anomeric linkage to the glycosyl moiety X;
   R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
   or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted.
   R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted.
b) Culturing said genetically modified cell in a culture medium comprising said exogenous precursor, whereby
   i. the exogenous precursor is internalized by the cell, and
   ii. one or more glycosylation reactions are performed on the internalized exogenous precursor or on a glycosylated derivative thereof by the one or more glycosyltransferases, to form the C-glycoside of interest,
c) Optionally isolating the C-glycoside of interest from the genetically modified cell and/or from the culture medium.

The method according to (1), wherein the genetically modified cell is a yeast cell or a bacterial cell, preferably an E. coli cell.

The method according to (1) or (2), wherein the one or more glycosyltransferase enzymes comprise one or more sialyltransferases and/or one or more fucosyltransferases, especially one or more sialyltransferases.

The method according to (1) or (2), wherein the one or more glycosyltransferase enzymes are selected from the group consisting of β-1,3-N-acetylglucosaminyltransferase, 0-1,6-N-acetylglucosaminyltransferase, β-1,3-galactosyltransferase, β-1,4-galactosyltransferase, β-1,4-N-acetylgalactosaminyltransferase, β-1,3-N-acetylgalactosaminyltransferase, β-1,3-glucoronosyltransferase, α-2,3-sialyltransferase, α-2,6-sialyltransferase, α-2,8-sialyltransferase, α-1,2-fucosyltransferase, α-1,3-fucosyltransferase, α-1,4-fucosyltransferase, α-1,4-galactosyltransferase, α-1,3-galactosyltransferase or a combination thereof.

The method according to any one of (1) to (4), wherein the genetically modified cell has no β-galactosidase activity.

The method according to any one of (1) to (5), wherein X of General Formula I is a monosaccharide moiety, a disaccharide moiety or a trisaccharide moiety, preferably a monosaccharide moiety or a disaccharide moiety.

The method according to any one of (1) to (5), wherein the exogenous precursor is a compound of General Formula Ia:

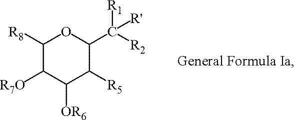

wherein
C, R′, R¹ and R² are as defined for General Formula I in (1),
R₅ is a group selected from OH, NH₂, NH-acyl,
R₆ and R₇ are independently selected from the group consisting of hydrogen or a glycosyl moiety and
R₈ is a group selected from CH₂—OH and C₁₋₆ alkyl, preferably methyl.

The method according to any one of (1) to (7), wherein the exogenous precursor is a compound of General Formula Ib:

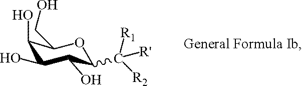

wherein
C, R′, R¹ and R² are as defined for General Formula I in (1),
and C-glycosidic bond

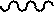

is preferably a β-C-glycosidic bond.

The method according to any one of (1) to (7), wherein the exogenous precursor is a compound of General Formula Ic:

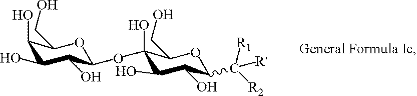

wherein
C, R′, R₁ and R₂ are as defined for General Formula I in (1),
and C-glycosidic bond

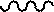

is preferably a β-C-glycosidic bond.

The method according to any one of (1) to (7), wherein the glycosylated C-glycoside of interest is a compound of General Formula IIa:

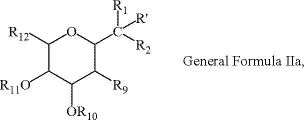

wherein
C, R′, R₁ and R₂ are as defined for General Formula I in (1),
R₉ is selected from the group consisting of OH, NH₂, NH-acyl and O-glycoside,
R₁₀ and R₁₁ are independently selected from the group consisting of hydrogen and a glycosyl moiety,
R₁₂ is selected from the group consisting of CH₂OH, CH₂O-glycoside and C₁₋₆ alkyl, preferably methyl.

The method according to (8), wherein the glycosylated C-glycoside of interest is a compound of General Formula IIb:

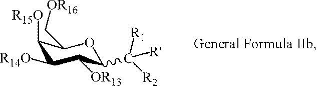

wherein
C, R′, R₁ and R₂ are as defined for General Formula I in (1),
R₁₃ to R₁₆ are independently selected from the group consisting of hydrogen and a glycosyl moiety.

The method according to (9), wherein the glycosylated C-glycoside of interest is a compound of General Formula IIc:

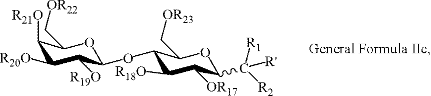

wherein
C, R′, R₁ and R₂ are as defined for General Formula I in (1),
R₁₇ to R₂₃ are independently selected from the group consisting of hydrogen and a glycosyl moiety.

The method according to (8), wherein the glycosyltransferase enzyme is a α-2,3-sialyltransferase and the produced glycosylated C-glycoside of interest is compound of General Formula IId or a salt thereof:

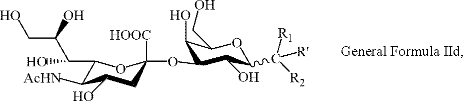

wherein
C, R′, R₁ and R₂ are as defined for General Formula I in (1),
and C-glycosidic bond

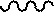

is preferably a β-C-glycosidic bond.

The method according to (8), wherein the glycosyltransferase enzymes are a α-2,3-sialyltransferase and a α-2,8-sialyltransferase and the produced glycosylated C-glycoside of interest is a compound of General Formula IIe or a salt thereof:

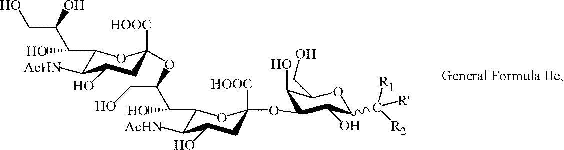

wherein
C, R′, R₁ and R₂ are as defined for General Formula I in (1),
and C-glycosidic bond

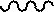

is preferably a β-C-glycosidic bond.

The method according to (9), wherein the glycosyltransferase enzyme is a α-2,3-sialyltransferase and the produced glycosylated C-glycoside of interest is a compound of General Formula IIf or a salt thereof:

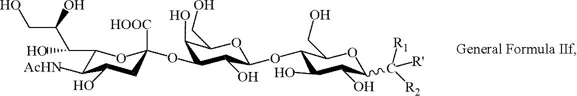

wherein
C, R′, R₁ and R₂ are as defined for General Formula I in (1),
and C-glycosidic bond

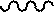

is preferably a β-C-glycosidic bond.

The method according to any one of (1) to (15), wherein R is acyl, preferably acetyl, and/or R₁ and R₂ are H.

Compound 2d or a salt thereof:

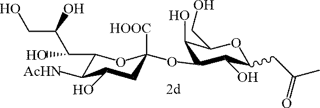

wherein C-glycosidic bond

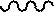

is preferably a β-C-glycosidic bond.

Compound 2e or a salt thereof:

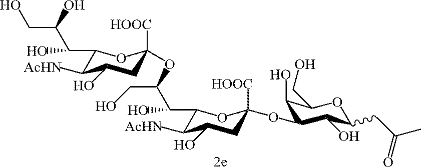

wherein C-glycosidic bond

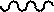

is preferably a β-C-glycosidic bond.

The present invention overcomes the drawbacks connected to the current synthesis techniques of C-glycosides and provides a new economically attractive method for the preparation of a broad variety of C-glycosides in cells expressing the required enzymes. This method enables the production of glycosyl fluorides having the desired stereo- and regiochemical configuration without the need for protecting group manipulations. Purification of the C-glycosides of interest can be achieved without the need for expensive and toxic reagents. Moreover, glycosyltransferases and glycosyl nucleotide donors may be produced by the engineered cell and are thus readily available. These advantages enable an easy scale-up production of complex C-glycosides.

### DETAILED DESCRIPTION OF THE INVENTION

The C-glycosides of interest of the present invention are produced starting from an exogenous precursor. The exogenous precursor is internalized by a cell that expresses one or more glycosyltransferases which catalyze the addition of further monosaccharide units to this exogenous precursor.

In the context of the present application, the following expressions are given a definition that should be taken into consideration with the claims and the description.

The term “substituted” means that the group in question is substituted with a group which typically modifies the general chemical characteristics of the group in question. Preferred substituents include but are not limited to halogen, nitro, amino, azido, oxo, hydroxyl, thiol, carboxy, carboxy ester, carboxamide, alkylamino, alkyldithio, alkylthio, alkoxy, acylamido, acyloxy, or acylthio, each of 1 to 6 carbon atoms, preferably of 1 to 3 carbon atoms. The substituents can be used to modify characteristics of the molecule as a whole such as molecule stability, molecule solubility and an ability of the molecule to form crystals. The person skilled in the art will be aware of other suitable substituents of a similar size and charge characteristics, which could be used as alternatives in a given situation.

In connection with the term “alkyl” and “aryl”, the term “optionally substituted” means that the group in question may be (is) substituted one or several times, preferably 1 to 3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono-and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The term “(hetero)alkyl chain” means an alkyl chain or a heteroalkyl chain.

The term “glycosyl moiety” when used herein is defined broadly to encompass a moiety derived from a monosaccharide unit or from an oligosaccharide, wherein the anomeric carbon of the monosaccharide or the anomeric carbon at the reducing end of the oligosaccharide is engaged in a glycosidic bond with another chemical entity. A glycosyl moiety having more than one monosaccharide may represent a linear or branched structure.

The monosaccharide unit can be any 5-9 carbon atom sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid).

The term “glycosyl moiety” for example includes the following moieties:

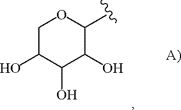

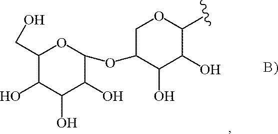

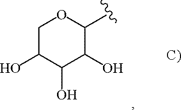

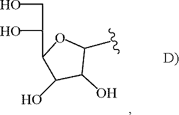

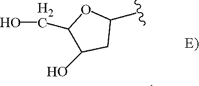

The term “nucleic acid sequence” refers to a DNA fragment, which is either double-stranded or single stranded, or to a product of transcription of said DNA fragment, and/or to an RNA fragment. A nucleic acid sequence may be naturally present in a cell where it is expressed (termed as “endogenous nucleic acid sequence”) or may be introduced into a cell by recombinant nucleic acid techniques (termed as “heterologous nucleic acid sequence”). Commonly known recombinant nucleic acid techniques are e.g. described in Sambrook et al., Molecular Cloning: A Laboratory Manual, 2^{nd} Ed., Cold Spring Harbor Laboratory Press, Cold Spring Harbor, N.Y. (1989). A heterologous nucleic acid sequence may be a nucleic acid sequence that originates from a source foreign to the particular host cell, or, if from the same source, is modified from its original form. Thus, a heterologous nucleic acid sequence in a cell also includes a nucleic acid sequence that is endogenous to the particular cell but has been subjected to one or more modifications. Modification of a nucleic acid sequence may occur, e.g., by treating the DNA with a restriction enzyme to generate a DNA fragment that is capable of being operably linked to a promoter. Techniques such as site-directed mutagenesis are also useful for modifying a nucleic acid sequence.

The exogenous precursor or exogenous precursor molecule is a C-glycoside represented by General Formula I, preferably by General Formula Ia, and more preferably by General Formula Ib or General Formula Ic as outlined in the present invention. The exogenous precursor molecule is modified by the method of the present invention in the way that one or more further monosaccharide units are attached to it by a glycosidic reaction. The C-glycoside of interest differs from the exogenous precursor in that the C-glycoside of interest comprises at least one more monosaccharide unit as compared to the exogenous precursor.

As the skilled person will understand, the term “C-glycoside” as used herein refers to a glycoside having a C-glycosidic linkage between a glycosyl moiety (“X” as used in the present invention) and a non-sugar moiety.

R₁ and R₂ as used in the General Formula presented herein are preferably H.

The C-glycosidic linkage according to the present invention linking a glycosyl moiety (outlined as “X” in the General Formulae represented herein) to a non-sugar moiety may be an α- or a β -C-glycosidic linkage. A β-C-glycosidic linkage is preferred.

The exogenous precursor can be synthesized by any method of producing C-glycosidic linkages known to a skilled person. The exogenous precursor is preferably synthesized chemically in a one-step procedure in alkaline aqueous media from unprotected carbohydrates via the Knoevenagel condensation as described e.g. in Rodrigues et al. 2000, Chem. Comm., 2049-2050.

The cell used in the method according to the present invention may be prokaryotic or eukaryotic. It may e.g. be a bacterial cell, a yeast cell or a mammalian cell. Preferably, the cell used in the method according to the present invention is a microorganism, such as a bacterium or a yeast. More preferably, the bacterium is selected from the group comprising Escherichia coli, Bacillus spp. (e.g. Bacillus subtilis), Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, Lactobacillus spp., Lactococcus spp., Enterococcus spp., Bifidobacterium spp., Sporolactobacillus spp., Micromomospora spp., Micrococcus spp., Rhodococcus spp. and Pseudomonas, and the yeast is selected from the group comprising Saccharomyces cerevisiae, Schizosaccharomyces pombe, Pichia pastoris and Candida albicans. Most preferably, the cell is an Escherichia coli (E. coli) cell.

The skilled person will understand that for the method of the present invention, the term “a genetically modified cell” does not intend to mean one single cell, but many cells, typically a cell clone showing the substantially the same genetic characteristics, that are cultured together in a culture medium. In the case of the cell originating from a mammal or from any other multicellular organism, the cells will be cultured in vitro isolated from the organism of origin. The expression “genetically modified” denotes that at least one alteration in the DNA sequence has been performed in the genome of the cell in order to give that cell a specific phenotype. The alteration in the DNA may e.g. be an introduction or a deletion of a DNA fragment in the genome. The alteration in the DNA sequence is herein especially achieved by the expression of a heterologous nucleic acid sequence, in particular a heterologous nucleic acid sequence encoding a glycosyltransferase enzyme. Genome editing may be performed e.g. by commonly known recombinant nucleic acid techniques as e.g. described in Sambrook et al., Molecular Cloning: A Laboratory Manual, 2^{nd} Ed., Cold Spring Harbor Laboratory Press, Cold Spring Harbor, N.Y. (1989). The CRISPR technology may also be used to perform genetic modifications.

The nucleic acid sequence encoding the glycosyltransferase enzyme may be an endogenous nucleic acid sequence or a heterologous nucleic acid sequence, preferably a heterologous nucleic acid sequence.

The genetically modified cell may comprise one or more than one nucleic acid sequence encoding one or more glycosyltransferase enzymes, such as two nucleic acid sequences encoding two or more glycosyltransferase enzymes, or three to five nucleic acid sequences encoding three to five glycosyltransferase enzymes. The nucleic acid sequences may be endogenous or heterologous. When more than one glycosyltransferase enzyme is expressed, the glycosyltransferase enzymes are preferably different ones and accordingly the encoding nucleic acid sequences are preferably different. When more than one glycosyltransferase enzyme is expressed and the encoding nucleic acid sequences are different, one or more encoding nucleic acid sequences may be endogenous.

In a preferred embodiment, the genetically modified cell further comprises one or more nucleic acid sequences encoding one or more epimerase enzymes, wherein the nucleic acid sequences may be endogenous or heterologous.

The origin of the heterologous nucleic acid sequences can be an animal (including humans), a plant, a yeast such as Saccharomyces cerevisiae, Saccharomyces pombe, Candida albicans, a bacterium such as E. coli, Bacillus subtilis, Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, a protozoa such as trypanosoma, or a virus.

The nucleic acid sequences according to the present invention comprise or are a gene, a derivative of a gene or a transcription product of a gene, or a synthetic construct substantially identical to a gene. A derivative of a gene includes a nucleic acid sequence that is a fragment of a gene or a nucleic acid sequence that contains one or more mutations and/or deletions as compared to the original gene, or a cDNA; the mutations or deletions must not strongly impair the function of the encoded enzyme. A derivative of a gene is preferably at least 60% identical to a gene, more preferably at least 90% identical to a gene, even more preferably at least 95% identical to a wildtype gene. The value for gene identity is typically generated when compared and aligned for maximum correspondence, as measured using one of the following sequence comparison algorithms or by visual inspection. A synthetic construct substantially identical to a gene may be produced by synthesis techniques known to the skilled person.

Because of the degeneracy of the genetic code, a large number of functionally identical nucleic acids encode any given peptide or protein. For instance, the codons CGU, CGC, CGA, CGG, AGA and AGG all encode the amino acid arginine. Thus, at every position where an arginine is specified by a codon, the codon can be altered to any of the corresponding codons described without altering the encoded peptide or protein. A derivative of a gene or a synthetic construct substantially identical to a gene is a nucleic acid sequence is in one embodiment codon-optimized for expression in the genetically modified cell according to the present invention.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel).

Examples of algorithms that are suitable for determining percent sequence identity and sequence similarity are the BLAST and BLAST 2.0 algorithms, which are described in Altschul el al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4, and a comparison of both strands.

In a preferred embodiment, one or more of the nucleic acid sequences according to the present invention are double-stranded DNA fragments. More preferably, the nucleic acid sequences according to the present invention are heterologous nucleic acid sequences. The heterologous nucleic acid sequence is may be placed in an expression cassette. The expression cassette comprises a promoter and the gene or a derivative thereof or synthetic construct to be transcribed. The promoter may be a constitutive or an inducible promoter. A preferred inducible promoter is the lac promoter. The promoter may be induced by addition of the inducer isopropyl β-D-thiogalactoside (IPTG) or by any other lactose analogue to the culture medium. Additional factors or effecting expression may also be used. Transcription start and termination signals, enhancers, and other DNA sequences that influence gene expression can also be included in an expression cassette. When more than one heterologous gene or derivative thereof or synthetic construct is expressed in the cell, the genes or derivatives thereof or synthetic constructs can be expressed on a single expression cassette or on multiple expression cassettes that are compatible and can be maintained in the same cell. When a single expression cassette is used for the expression of more than one heterologous gene or derivative thereof or synthetic construct, the heterologous genes or derivatives thereof or synthetic constructs may be placed under the same promoter, such as an operon, or under several promoters. When several promoters present in one or more expression cassettes are used for the expression of several heterologous genes or derivatives thereof of synthetic constructs, these promoters may be identical or different. Several different inducible promoters present in one or more expression cassettes may be induced by different inducers.

The expression cassette may in one embodiment be introduced into the cell by being placed on an expression vector. The expression vector typically further comprises a selection marker, including e.g. ampicillin or kanamycin.

A heterologous nucleic acid sequence can be expressed in the cell transiently or stably. For example, one expression vector can be used for one or several expression cassettes or more than one expression vector can be used for more than one expression cassette. Heterologous nucleic acid sequences according to the present invention can also be inserted into the chromosome of the cell, using methods known to those skilled in the art, including homologous recombination, site-specific recombination or transposon-mediated gene transposition. The CRISPR technology may also be used to insert one or more heterologous nucleic acid sequences or one or more expression cassettes into a specific locus of the chromosome of the cell. Combinations of expression cassettes in extrachromosomal vectors and expression cassettes inserted into a host cell chromosome can also be used.

Glycosyltransferases are enzymes that catalyze glycosylation reactions between a glycosyl donor, which is typically an activated sugar nucleotide (for Leloir glycosyltransferases), and a glycosyl acceptor, which is a nucleophilic biomolecule including a sugar, a protein or a lipid. Activated sugar nucleotides generally comprise a phosphorylated glycosyl residue attached to a nucleoside. The glycosyl residue of the donor is transferred to the acceptor by a glycosyltransferase, forming a glycosidic linkage.

The glycosyltransferase enzyme encoded by the nucleic acid sequence according to the method of the present invention is typically a Leloir glycosyltransferase, capable of performing a glycosylation reaction between the exogenous precursor and an activated sugar nucleotide. The glycosyltransferase enzyme(s) according to the method of the present invention may be a glucosyltransferase, a galactosyltransferase, an N-acetylglucosaminyltransferase, an N-acetylgalactosaminyltransferase, a glucoronosyltransferase, a xylosyltransferase, a mannosyltransferase, a fucosyltransferase, a sialyltransferase, and the like. In a preferred embodiment, the glycosyltransferase enzyme encoded by the nucleic acid sequence according to the method of the present invention may be an α-2-0-fucosyltransferase, a β-1,3-N-acetylglucosaminyltransferase, a β-1,6-N-acetylglucosaminyltransferase, a β-1,3-galactosyltransferase, a β-1,4-galactosyltransferase, a β-1,4-N-acetylgalactosaminyltransferase, β-1,3-N-acetylgalactosaminyltransferase, a β-1,3-glucoronosyltransferase, an α-2,3-sialyltransferase, an α-2,6-sialyltransferase, an α-2,8-sialyltransferase, an α-1,2-fucosyltransferase, an α-1,3-fucosyltransferase, an α-1,4-fucosyltransferase, an α-1,4-galactosyltransferase, an α-1,3-galactosyltransferase, or a combination thereof.

In some preferred embodiments, the one or more glycosyltransferase enzymes encoded by the one or more nucleic acid sequences according to the method of the present invention comprise one or more sialyltransferases (EC 2.4.99.-). In some more preferred embodiments, the one or more sialyltransferases comprise an α-2,3-sialyltransferase (β-galactoside α-2,3-sialyltransferase (EC 2.44.99.4)), an α-2,6-sialyltransferase (β-galactoside α-2,6-sialyltransferase (EC 2.44.99.1), an α-2,8-sialyltransferase (α-N-acetylneuraminate α-2,8-sialyltransferase (EC 2.44.99.8), or a combination thereof.

In some preferred embodiments, the one or more glycosyltransferase enzymes encoded by the one or more nucleic acid sequences according to the method of the present invention comprise one or more fucosyltransferases. In some more preferred embodiments, the one or more fucosyltransferases comprise an α-1,2-fucosyltransferase (type 1 galactoside α-1,2-fucosyltransferase (EC 2.4.1.69)), an α-1,3-fucosyltransferase (glycoprotein 3- α-L-fucosyltransferase (EC 2.4.1.214), an α-1,4-fucosyltransferase (EC 2.4.1.65), or a combination thereof.

In some preferred embodiments, the one or more glycosyltransferase enzymes encoded by the one or more nucleic acid sequences according to the method of the present invention comprise one or more fucosyltransferases and one or more sialyltransferases.

In a more preferred embodiment, the glycosyltransferase enzyme encoded by the nucleic acid sequence according to the present invention is an α-2,3-sialyltransferase and/or an α-2,8-sialyltransferase.

In an especially preferred first embodiment, the glycosyltransferase enzyme encoded by the nucleic acid sequence according to the present invention is α-2,3-sialyltransferase. The nucleic acid sequence according to the present invention encoding the α-2,3-sialyltransferase may be the gene nst from Neisseria meningitidis (N. meningitidis) (GenBank accession number U60660).

In an especially preferred second embodiment, the glycosyltransferase enzymes encoded by the nucleic acid sequences according to the present invention are an α-2,3-sialyltransferase and an α-2,8-sialyltransferase. The nucleic acid sequence according to the present invention encoding the α-2,3-sialyltransferase and α-2,8-sialyltransferase, respectively, may be the gene cstII from Campylobacter jejuni (C. jejuni) encoding the bifunctional α-2,3- and α-2,8-sialyltransferase (GenBank accession number AF400048).

The activated sugar nucleotide used for the glycosylation reaction of the present invention may e.g. be UDP-Glc, UDP-Gal, UDP-GlcNAc, UDP-GalNAc, UDP-glucuronic acid, UDP-Xyl, GDP-Man, GDP-Fuc and CMP-sialic acid. The activated sugar nucleotide used for the glycosylation reaction of the present invention is preferably CMP-sialic acid. A skilled person knows that the choice of glycosyltransferase enzyme determines the sugar nucleotide possible as donor for the glycosylation reaction. When more than one different glycosyltransferase enzyme is expressed in the cell, also more than one different activated sugar nucleotide may be needed to be present in the cell, depending on whether the different glycosyltransferases use the same or different sugar nucleotides as donors.

The activated sugar nucleotide is typically synthesized by a suitable nucleotidylyltransferase from a carbon substrate. Accordingly, the genetically modified cell used for the method of the present invention preferably comprises a nucleic acid sequence encoding a nucleotidylyltransferase capable of producing the desired activated sugar nucleotide. The nucleic acid sequence may be naturally present in the cell or may be heterologously expressed after introduction into the cell by means of recombinant techniques generally known to the skilled person. Preferred nucleotidylyltransferases include uridylyltransferases, guanylyltransferases and cytitidylyltransferases. The carbon substrate may be used from an exogenous addition to the genetically modified cell and/or may originate from a salvage pathway. Preferred carbon substrates include glycerol, glucose, glycogen, fructose, maltose, starch, cellulose, pectin, sucrose or chitin.

When an α-2,3-sialyltransferase and/or an α-2,8-sialyltransferase are expressed in the genetically modified cell according to the present invention, CMP-sialic acid is typically used as donor for the glycosylation reactions on the exogenous precursor and on a glycosylated derivative thereof, respectively. CMP sialic acid may be produced in the cell from UDP-GlcNAc by the expression of genes encoding a CMP-Neu5Ac synthetase, a sialic acid synthase and a GlcNAc-6-phosphate 2 epimerase while eliminating the activity of N-acetylmannosamine (ManNAc) kinase and N-acetyl-D-neuraminic acid (Neu5Ac) aldolase. The CMP-Neu5Ac synthetase is preferably encoded by the gene neuA from Campylobacter jejuni (AF400048), the sialic acid synthase is preferably encoded by the gene neuB from C. jejuni (AF400048) and the GlcNAc-6-phosphate 2 epimerase is preferably encoded by the gene neuC from C. jejuni (AF400048). The genes may be heterologously expressed in the genetically modified cell of the present invention, while, where e.g. E. coli is the genetically modified cell, the nanKETA genes have been inactivated.

In step b) of the method of the present invention, the genetically modified cell is cultured in a culture medium. When the cell of the present invention is a bacterial or a yeast cell, the culturing corresponds to a fermentation process and the “culture medium” may be also termed as “fermentation broth”.

A fermentation process typically includes two phases:
1. a first phase of exponential cell growth ensured by a carbon-based substrate, and
2. a second phase of cell growth limited by a carbon-based substrate which is added continuously.

The fermentation process may preferably further comprise a third phase (3.) of slowed cell growth obtained by continuously adding to the culture an amount of carbon-based substrate that is less than the amount of carbon-based substrate added in the second phase of the fermentation process so as to further increase the produced compound. More preferably, the amount of carbon-based substrate added during the third phase of the fermentation is at least 30% less than the amount of the carbon-based substrate added during the second phase of the fermentation.

During fermentation, the exogenous precursor may be added to the culture medium at one time point, stepwise or continuously. The pure precursor in solid or in liquid form or a concentrated aqueous solution of the precursor can be added at one time point at the start of fermentation or at the end of the first phase of exponential growth.

The carbon-based substrate may be selected from sucrose, glycerol and glucose. The carbon-based substrate added during the second phase is preferably glycerol.

The culturing is preferably performed under conditions allowing the production of a culture with a high cell density. The skilled person is aware of such conditions, including e.g. pH control and pO₂ control. pO₂ is preferably more than 10%, more preferably more than 20%, even more preferably more than 40% with air flow and stirring.

The first phase of the fermentation process may be performed at a reaction temperature of e.g. 30° C., 31° C., 32° C., 33° C., 34° C., 35° C. or 36° C.

The second phase of the fermentation process may be performed at a reaction temperature of e.g. 25° C., 26° C., 27° C., 28° C., 29° C. or 30° C.

The pH regulated may be kept stable by the addition of e.g. aqueous NH₄OH, NaOH or KOH solution.

In step b) i. of the method of the present invention, the exogenous precursor is internalized by the cell. The internalization step should not affect the basic and vital functions or destroy the integrity of the cell. The exogenous precursor molecule may be internalized solely or also via a passive transport during which the exogenous precursor molecule diffuses passively across the plasma membrane of the cell. The flow is directed by the concentration difference in the extra- and intracellular space with respect to the exogenous precursor molecule to be internalized, which exogenous precursor molecule is supposed to pass from the place of higher concentration to the zone of lower concentration tending towards an equilibrium.

Typically, the genetically modified cell comprises a transporter protein, which internalizes the exogenous precursor molecule via active transport. Different transporter proteins have specificities for different sugar moieties of the molecules to be internalized. This specificity may be altered by mutation by means of common recombinant DNA techniques. Preferably, the internalization of the exogenous precursor molecule is performed via a transporter protein.

The internalized precursor is then subjected to a glycosylation reaction according to step b) ii) of the method of the present invention. For the glycosylation reaction of the present invention taking place in the cell, the exogenous precursor molecule serves as glycosyl acceptor. The addition of one monosaccharide unit to the exogenous precursor molecule is performed by a glycosyltransferase. The resulting molecule is termed in the present context as “glycosylated derivative of the exogenous precursor” or as “C-glycoside of interest”, depending on whether the molecule is subjected to at least one further glycosylation reaction in the cell (then termed as “glycosylated derivative of the exogenous precursor” or just “glycosylation derivative”) or whether it is the final molecule to be produced and subjected to step c) of the method of the present invention (then termed as “C-glycoside of interest”). If more than one glycosylation reaction is performed in the cell, the “glycosylation derivative” is the acceptor molecule for the second and every further glycosylation reaction. One to five glycosylation reactions are preferably performed in the cell. The monosaccharide units that are added during a second and any further glycosylation reaction may be identical or different. The skilled person will understand that the addition of different monosaccharide units is performed by different glycosyltransferases, which are encoded by different nucleic acid sequences, using different activated sugar nucleotides as donor molecule. The addition of identical monosaccharide units is typically also performed by different glycosyltransferases, which are encoded by different nucleic acid sequences, using however the same activated sugar nucleotides as donor molecule. Accordingly, when the C-glycoside of interest comprises at least two more monosaccharide units as compared to the exogenous precursor, those monosaccharide units are either identical or different from each other.

The exogenous precursor is a compound of General Formula I

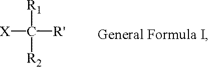

wherein
X represents a glycosyl moiety;
C is a carbon atom connected via anomeric linkage to the glycosyl moiety X;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted,
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted.
Glycosyl moiety X of General Formula I is in a preferred embodiment a monosaccharide moiety, a disaccharide moiety or a trisaccharide moiety, more preferably a monosaccharide moiety or a disaccharide moiety. Glycosyl moiety X of General Formula I is in a most preferred embodiment a disaccharide moiety.

In one preferred embodiment, the exogenous precursor is compound 1b:

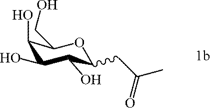

Wherein the C-glycosidic bond

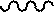

is preferably a β-C-glycosidic bond.

In another preferred embodiment the exogenous precursor is compound 1c:

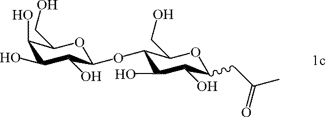

Wherein the C-glycosidic bond

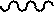

is preferably a β-C-glycosidic bond.

The skilled person will understand that the preferred embodiments as mentioned for the exogenous precursor, with the exception of the glycosyl moiety, equally relate to the C-glycoside of interest, the latter ones being produced from the former ones.

In some embodiments, the glycosyl moiety of the C-glycoside of interest corresponds to the glycosyl moiety of a ganglioside, such as GM1a, GM1b, GM2, GM3, GD3, GM4, GD1a, GD1b.

In some embodiments, the glycosyl moiety of the C-glycoside of interest corresponds to the glycosyl moiety of a human milk oligosaccharide (HMO), such as LNT, LNnT, LNH, LNnH, 2′FL, 3′FL, DFL, LNFP-I, LNFP-II, 3′SL, 6′SL.

The C-glycoside of interest is preferably selected from 3′-α-N-acetylneuroaminoyl-(2→3)-β-D-galactopyranosyl-2′-propanone (2g), 3′-α-N-acetylneuroaminoyl-(2→8)-α-N-acetylneuroaminoyl-(2→3)-β-D-galactopyranosyl-2′-propanone (2h), 3′-α-N-acetylneuroaminoyl-(2→3)-β-D-galactopyranosyl-(1→4)-(β-D-glucopyranosyl)-2′-propanone (2i) and 3′α-N-acetylneuroaminoyl-(2→8)-α-N-acetylneuroaminoyl-(2→3)-β-D-galactopyranosyl-(1→4)-(β-D-glucopyranosyl)-2′-propanone (2j):

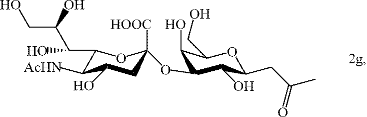

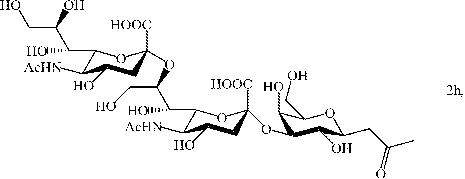

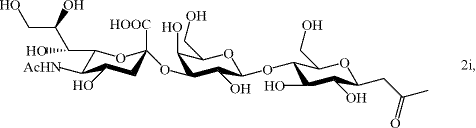

and

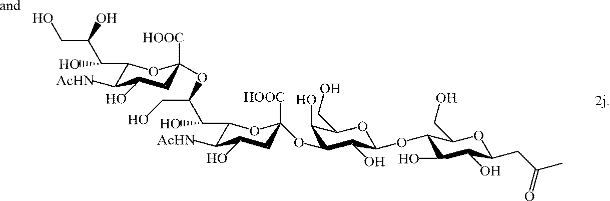

In a preferred embodiment, the genetically modified cell lacks any enzymatic activity which would degrade the exogenous precursor, the glycosylated derivatives of the exogenous precursor and/or the C-glycoside of interest. In a more preferred embodiment, the endogenous gene encoding for β-galactosidase (EC 3.2.1.23), the endogenous gene encoding for N-acetylmannosamine kinase (EC 2.7.1.60) or especially the endogenous genes encoding for β-galactosidase and for N-acetylmannosamine is/are inactivated in the genetically modified cell, so as no functional enzyme can be produced. Most preferably, the endogenous gene encoding for β-galactosidase is inactivated in the genetically modified cell. Accordingly, where the genetically modified cell is E. coli, the gene lacZ is preferably inactivated. The gene encoding for α-galactosidase (in E. coli the gene melA) may also be inactivated.

Step c) of the method of the present invention relates to the isolation of the C-glycoside of interest from the cell or from the culture medium. Step c) may as well be an optional step of the method according to the present invention. The C-glycoside of interest of the method of the present invention can accumulate both in the intra- and extracellular matrix. C-glycosides having more monosaccharide units tend to accumulate in the cell, while C-glycosides having less monosaccharide units are rather exported from the cell. When exported, the C-glycoside of interest may be exported from the cell via passive transport, by diffusing outside across the cell membrane into the culture medium. The export may be facilitated or mediated by sugar efflux transporters. A sugar efflux transporter may be naturally present in the cell or may be provided in form of a heterologous nucleic acid sequence encoding for the sugar efflux transporter produced by recombinant techniques known to the skilled person. The endogenous or heterologous nucleic acid sequence encoding for the sugar efflux transporter may in one embodiment be mutated by means of known recombinant techniques or may be overexpressed to increase the specificity towards the carbohydrate moiety of the C-glycoside of interest to be secreted.

For the isolation step, the culture medium is preferably separated from the cells by filtration or centrifugation. When the C-glycoside of interest is mainly exported from the cell, it is mainly present in the supernatant containing the culture medium and purified and isolated therefrom by means of standard separation, purification and isolation techniques such as crystallization, precipitation and chromatography (e.g. silica, reverse phase, size exclusion, gel and/or cation ion exchange resin chromatography, etc.). When the C-glycoside of interest accumulates mainly inside the cell, the separated cells are preferably permeabilized. For that, the cells are resuspended in water and subjected to heat and/or acid or base treatment. Sodium hydroxide may be used for a base treatment and sulfuric acid may be used for acid treatment. The C-glycoside of interest is then separated from the treated cells by filtration and purified and isolated from the supernatant by means of standard separation, purification and isolation techniques such as gel and/or cation ion exchange resin chromatography. The supernatant containing the product from the culture medium may in one embodiment be combined with the supernatant containing the product from the lysed cells. Also in this embodiment, the product may be purified and isolated from the combined supernatant by means of standard separation, purification and isolation techniques such as gel and/or ion exchange resin chromatography.

The invention relates in one preferred embodiment to a method for producing 3′-α-N-acetylneuroaminoyl-(2-3)-α/β-D-galactopyranosyl-2′-propanone (2d), the method comprising the steps of:
a) Providing an exogenous precursor and a genetically modified cell, wherein the exogenous precursor is 3′-(a/β-D-galactopyranosyl)-2′-propanone (1b), preferably 3′-(β-D-galactopyranosyl)-2′-propanone (1d), and wherein the genetically modified cell is an E. coli cell comprising a heterologous nucleic acid sequence encoding an α-2,3-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli cell have been inactivated;
b) Culturing said genetically modified cell in a culture medium comprising said exogenous precursor, whereby
   i. the exogenous precursor is internalized by the cell, and
   ii. one glycosylation reaction is performed on the internalized exogenous precursor, to form 2d,
c) Optionally isolating the 2d from the genetically modified cell and/or from the culture medium.

The invention relates in another preferred embodiment to a method for producing 3′-α-N-acetylneuroaminoyl-(2→8)-α-N-acetylneuroaminoyl-(2→3)-α/β-D-galactopyranosyl-2′-propanone (2e), the method comprising the steps of:
a) Providing an exogenous precursor and a genetically modified cell, wherein the exogenous precursor is 3′-(a/β-D-galactopyranosyl)-2′-propanone (1b), preferably 3′-(β-D-galactopyranosyl)-2′-propanone (1d), and wherein the genetically modified cell is an E. coli cell comprising a heterologous nucleic acid sequence encoding an α-2,3-sialyltransferase and an α-2,8-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli cell have been inactivated;
b) Culturing said genetically modified cell in a culture medium comprising said exogenous precursor, whereby
   i. the exogenous precursor is internalized by the cell, and
   ii. one glycosylation reaction is performed on the internalized exogenous precursor, to form 2e,
c) Optionally isolating the 2e from the genetically modified cell and/or from the culture medium.

The invention relates in another preferred embodiment to a method for producing 3′-α-N-acetylneuroaminoyl-(2-3)-β-D-galactopyranosyl-(1-4)-α/β-D-glucopyranosyl-2′-propanone (2f)

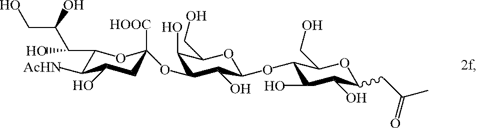

the method comprising the steps of:
a) Providing an exogenous precursor and a genetically modified cell, wherein the exogenous precursor is 3′-(α/β-D-lactosyl)-2′-propanone (1c), preferably 3′-(β-D-lactosyl)-2′-propanone (1e), and wherein the genetically modified cell is an E. coli 1acZ⁻ cell comprising a heterologous nucleic acid sequence encoding an α-2,3-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli 1acZ⁻ cell have been inactivated;
b) Culturing said genetically modified cell in a culture medium comprising said exogenous precursor, whereby
   i. the exogenous precursor is internalized by the cell, and
   ii. one glycosylation reaction is performed on the internalized exogenous precursor, to form 2f,
c) Optionally isolating the 2f from the genetically modified cell and/or from the culture medium.

### EXAMPLES

### Example 1: Preparation of the Genetically Engineered Bacterial Strain

The engineered E. coli host strain used for fermentation and the transformed plasmids were constructed in accordance with WO 2007/101862 A1, Fierfort et al. Journal of Biotechnology 134, 261-265 (2008) and Priem et al. Glycobiology 12(4), 234-240 (2002); the strain was engineered from an E. coli K12 strain derivative in which the genes lacA and lacZ as well as the genes nanKETA have been deleted and which has been co-transformed with a plasmid carrying the neuABC genes from Campylobacter jejuni, and a second plasmid carrying the α-2,3-sialyltransferase-encoding nst gene from Neisseria meningitidis for the production of 2g or 2i with a second plasmid carrying the α-2,3 α-2,8-sialyltransferase-encoding cstII gene from Campylobacter jejuni for the production of 2h or 2j, respectively.

### Example 2 Synthesis of 3′-(β-D-galactopyranosyl)-2′-propanone (1d)

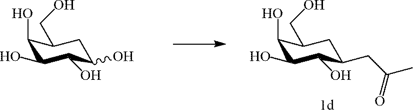

Galactose (1.8 g; 10 mmol) was dissolved in 40 mL water. NaHCO₃ (1.26 g; 15 mmol) and acetylacetone (1.23 mL; 12 mmol) was added. The mixture was stirred at 90° C. for 20 hours. The solution is cooled down to room temperature and neutralized with IR 120 resin. The resin was filtrated out and the filtrate was concentrated under vacuum. The product was crystallized from methanol to yield 1d (1.1 g) as an off-white crystalline solid.

¹H NMR (400 MHz, D₂O) δ = 3.91 (d, J = 3.4 Hz, 1H), 3.73 - 3.55 (m, 5H), 3.41 (dd, J = 9.6, 9.6 Hz, 1H), 2.98 (dd, J = 16.7, 3.0 Hz, 1H), 2.70 (dd, J = 16.7, 9.2 Hz, 1H), 2.23 (s, 3H) ppm.

¹³C NMR (101 MHz, D₂O) δ = 213.33, 78.52, 75.61, 73.74, 70.38, 69.03, 61.11, 45.69, 29.72 ppm.

### Example 3 Synthesis of 3′-(β-D-lactosyl)-2′-propanone (le)

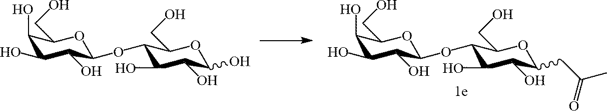

Lactose (3.6 g; 10 mmol) was dissolved in 40 mL water. NaHCO₃ (1.26 g; 15 mmol) and acetylacetone (1.23 mL; 12 mmol) was added. The mixture was stirred at 90° C. for 20 hours. The solution is cooled down to room temperature and neutralized with IR 120 resin. The resin was filtrated out and the filtrate was concentrated under vacuum. The product was crystallized from methanol to yield 1e (2.13 g) as an off-white crystalline solid.

¹H NMR (D₂O, 400 MHz): 4.39 (d, 1H, J = 8), 3.88-3.83 (m, 2H), 3.78-3.55 (m, 8H), 3.51-3.49 (bt, 2H), 3.23 (bt, 1H), 2.96 (dd, 1H, J₁ = 16, J₂ = 4), 2.67 (dd, 1H, J₁ = 16, J₂ = 8), 2.22 (s, 3H).

¹³C (D₂O, 100 MHz): 213.22, 102.87, 78.45, 78.34, 75.76, 75.35, 75.08, 72.77, 72.53, 70.96, 68.56, 61.03, 60.09, 45.55, 29.83.

### Example 4: Fermentation Procedure

The culture was carried out in a 21 fermenter containing 1 liter of minimal medium containing ammonium phosphate 87 mM, potassium phosphate 51 mM, TMS-A, Citric Acid 5.2 mM, potassium hydroxide 45 mM, sodium hydroxide 25 mM, magnesium sulphate 2.5 mM as well as Glucose 15.9 g/L and Glycerol 2.4 g/L as initial carbon source. The growth phase started with the inoculation (2% inoculum). The temperature was kept at around 33° C. and the pH regulated at 6.8 with aqueous NH₄OH solution. The oxygen was kept at 40% with an air flow between 0.5 to 3 L/min until cells were adapted to the glycerol in the medium. When all initial carbon source was depleted, the fed-batch phase was initiated, the exogenous precursor 3′-(β-D-galactopyranosyl)-2′-propanone (1d, 68 mM) or 3′-(β-D-lactosyl)-2′-propanone (1e, 65.4 mM) and the inducer IPTG (1-2 ml of a 50 ng/ml solution) were added to the culture and the temperature was decreased to 28° C. The fed-batch was realized using a 750 g/L aqueous glycerol solution, with a high substrate feeding rate of ≈4.5 g/h of glycerol for 11 of culture. The culture was monitored by HPLC, and the identity of the peaks was confirmed by MS analysis (for the method see example 9). The maximal production yield for the fermentation was observed after 24 h for compounds 2g and 2i and 92 h for compound 2h.

### Example 5: General Purification Procedure

The fermentation broth was ultrafiltered (5-30 kDa membrane) at 25° C. until the total volume was concentrated to half and the UF permeate was collected. The UF retentate was then washed with purified water (4 to 5-fold volumes relative to the ultrafiltered broth volume) until all compound of interest was extracted to the permeate. The combined UF permeates were then subjected to nanofiltration (300-500 Da membrane) at 30 bar and 15° C. until the retentate reached a concentration 20 to 30-fold higher than the initial solution.

The NF retentate was subjected to standard chromatographic techniques to afford the final compound.

### Example 6: 3′-α-N-acetylneuroaminoyl-(2→3)-β-D-galactopyranosyl-2′-propanone (2g)

C-glycoside 2g was obtained following the general fermentation and purification procedures described in examples 4 and 5. MS: 512 Da [M+H]⁺

### Example 7: 3′-α-N-acetylneuroaminoyl-(2→8)-α-N-acetylneuroaminoyl -(2→3)-β-D-galactopyranosyl-2′-propanone (2h)

C-glycoside 2h was obtained following the general fermentation and purification procedures described in examples 4 and 5. MS: 803 Da [M+H]⁺

### Example 8: 3′-α-N-acetylneuroaminoyl-(2→3)-β-D-galactopyranosyl-(1→4)-β-D-glucopyranosyl-2′-propanone (2i)

C-glycoside 2i was obtained following the general fermentation and purification procedures described in examples 4 and 5. MS: 674.1 Da [M+H]⁺

### Example 9: 3′ α-N-acetylneuroaminoyl-(2→8)-α-N-acetylneuroaminoyl-(2→3)-B-D-galactopyranosyl-(1→4)-(β-D-glucopyranosyl)-2′-propanone (2j)

C-glycoside 2j was obtained following the general fermentation and purification procedures described in examples 4 and 5. MS: 965.2 Da [M+H]⁺

### Example 10: MS Analysis

The mass spectrometer was scanned in TOF mode with accumulation time of 1 sec. Mass range was 300-3000 amu.

1. Method for producing a C-glycoside of interest, the method comprising the steps of:
a) Providing an exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes, and wherein the exogenous precursor is a compound of General Formula I 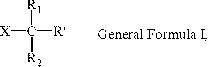
wherein
X represents a glycosyl moiety;
C is a carbon atom connected via anomeric linkage to the glycosyl moiety X;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted.
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted.
b) Culturing said genetically modified cell in a culture medium comprising said exogenous precursor, whereby
i. the exogenous precursor is internalized by the cell, and
ii. one or more glycosylation reactions are performed on the internalized exogenous precursor or on a glycosylated derivative thereof by the one or more glycosyltransferases, to form the C-glycoside of interest,
c) Optionally isolating the C-glycoside of interest from the genetically modified cell and/or from the culture medium.

2. The method according to claim 1, wherein the genetically modified cell is a yeast cell or a bacterial cell, .

3. The method according to claim 1, wherein the one or more glycosyltransferase enzymes comprise one or more sialyltransferases and/or one or more fucosyltransferases, especially one or more sialyltransferases.

4. The method according to claim 1, wherein the one or more glycosyltransferase enzymes are selected from the group consisting of β-1,3-N-acetylglucosaminyltransferase, (β-1,6-N-acetylglucosaminyltransferase, β-1,3-galactosyltransferase, β-1,4-galactosyltransferase, β-1,4-N-acetylgalactosaminyltransferase, β-1,3-N-acetylgalactosaminyltransferase, β-1,3-glucoronosyltransferase, α-2,3-sialyltransferase, α-2,6-sialyltransferase, α-2,8-sialyltransferase, α-1,2-fucosyltransferase, α-1,3-fucosyltransferase, α-1,4-fucosyltransferase, α-1,4-galactosyltransferase, α-1,3-galactosyltransferase or a combination thereof.

5. The method according to claim 1, wherein the genetically modified cell has no β-galactosidase activity.

6. The method according to claim 1, wherein X of General Formula I is a monosaccharide moiety, a disaccharide moiety or a trisaccharide moiety .

7. The method according to claim 1, wherein the exogenous precursor is a compound of General Formula Ia: 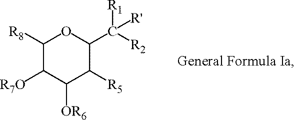
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₅ is a group selected from OH, NH₂, NH-acyl;
R₆ and R₇ are independently selected from the group consisting of hydrogen or a glycosyl moiety and
R₈ is a group selected from CH₂—OH and C₁-₆ alkyl.

8. The method according to claim 1, wherein the exogenous precursor is a compound of General Formula Ib: 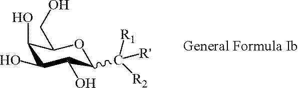
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted .

9. The method according to claim 1, wherein the exogenous precursor is a compound of General Formula Ic: 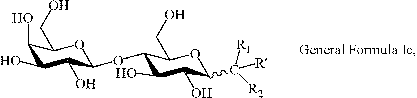
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted .

10. The method according to claim 1, wherein the glycosylated C-glycoside of interest is a compound of General Formula IIa: 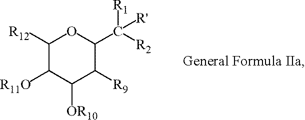
wherein
C is a carbon atom connected via anomeric linkage to the glycosyl moiety X;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted; R₉ is selected from the group consisting of OH, NH₂, NH-acyl and O-glycoside, R₁₀ and R₁₁ are independently selected from the group consisting of hydrogen and a glycosyl moiety,
R₁₂ is selected from the group consisting of CH₂OH, CH₂O-glycoside and C₁₋₆ alkyl .

11. The method according to claim 8, wherein the glycosylated C-glycoside of interest is a compound of General Formula IIb: 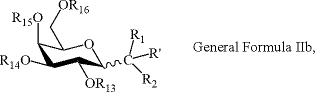
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted; R₁₃, R₁₄, R₁₅, R₁₆ are independently selected from the group consisting of hydrogen and a glycosyl moiety.

12. The method according to claim 9, wherein the glycosylated C-glycoside of interest is a compound of General Formula IIc: 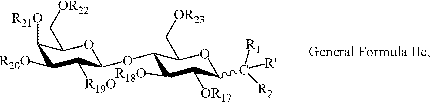
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted; R₁₇, R₁₈, R₁₉, R₂₀, R₂₁, R₂₂ R₂₃ are independently selected from the group consisting of hydrogen and a glycosyl moiety.

13. The method according to claim 8, wherein the glycosyltransferase enzyme is a α-2,3-sialyltransferase and the produced glycosylated C-glycoside of interest is compound of General Formula IId or a salt thereof: 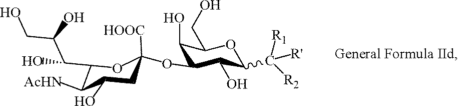
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted .

14. The method according to claim 8, wherein the glycosyltransferase enzymes are a α-2,3-sialyltransferase and a α-2,8-sialyltransferase and the produced glycosylated C-glycoside of interest is compound of General Formula IIe or a salt thereof: 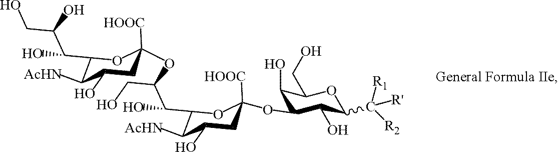
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted .

15. The method according to claim 9, wherein the glycosyltransferase enzyme is a α-2,3-sialyltransferase and the produced glycosylated C-glycoside of interest is a compound of General Formula IIf or a salt thereof: 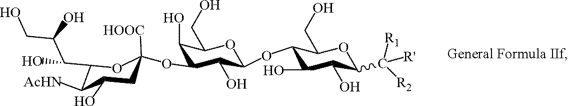
wherein
C is a carbon atom;
R′ is selected from the group consisting of H, vinyl, allyl, ethynyl, cycloalkyl or heterocycloalkyl, aryl or heteroaryl, or of a (hetero)alkyl chain which may be straight or branched and/or which may be saturated or contain one or more double and/or triple bonds, wherein said vinyl, allyl and ethynyl, said cycloalkyl or heterocycloalkyl, said aryl or heteroaryl and said (hetero)alkyl chain may be substituted or unsubstituted;
or R′ represents a substituent selected from azido, cyano, halogen, OR₃, SR₃, NR₃R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted;
R₁ and R₂ are independently selected from the group consisting of H, saturated or unsaturated alkyl, aryl, cycloalkyl, vinyl, allyl, ethynyl, each of which may be substituted or unsubstituted, and/or R₁ and R₂ independently represent a substituent selected from azido, cyano, OR₃, SR₃, NR₃ R₄, COR₃, COOR₃, CONR₃R₄, wherein R₃ and R₄ are independently selected from the group consisting of H, aryl, saturated or unsaturated alkyl, vinyl, allyl, ethynyl, cycloalkyl, each of which may be substituted or unsubstituted .

16. The method according to claim 1, wherein R′ is acyl and/or R¹ and R² are H.

17. A compound 2d or a salt thereof: 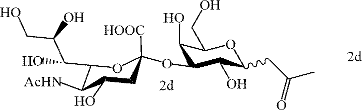
.

18. A compound 2e or a salt thereof: 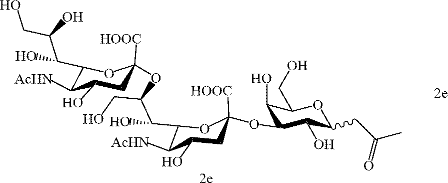
.
